(19) United States
(12) Patent Application Publication
(10) Pub. No.: US 2023/0097009 A1
(43) Pub. Date: Mar. 30, 2023

(54) USE OF COMPOUND IN PREVENTION AND/OR TREATMENT OF PATHOGEN INFECTION IN ANIMALS

(71) Applicant: FELICAMED BIOTECHNOLOGY CO., LTD., Zhuhai, Guangdong (CN)

(72) Inventors: Di KANG, Zhuhai, Guangdong (CN); Danni LI, Zhuhai, Guangdong (CN); Xingyu LIN, Zhuhai, Guangdong (CN); Tingting LU, Zhuhai, Guangdong (CN)

(21) Appl. No.: 17/801,219

(22) PCT Filed: Jun. 11, 2021
(86) PCT No.: PCT/CN2021/099650

§ 371 Date: Aug. 19, 2022

(30) Foreign Application Priority Data
Jun. 16, 2020 (CN) 202010547186.8

(51) Int. Cl. A61K 31/444; A61P 31/00

(52) U.S. Cl. CPC A61K 31/444; A61P 31/00

(57) ABSTRACT

Disclosed is use of a compound in preventing and/or treating pathogen infection in animals. The compound is 4-(3-(((2-amino-5-(2-(1-methylpiperidin-4-yl)thiazol-5-yppyridin-3-ypoxy)methyl) phenyl)-2-methylbut-3-yn-2-ol, having a structure of formula I below. The compound can effectively treat diseases (such as feline infectious peritonitis) caused by pathogen (especially virus, such as coronavirus) infection in animals, improves the survival conditions of the animals, and has better commercial value and application prospect in the field of veterinary anti-pathogen (especially virus, such as coronavirus) therapies.

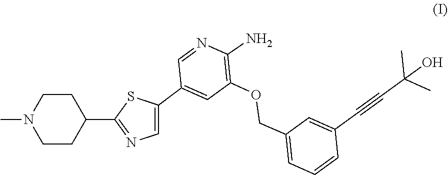 [SMILES: CN1CCC(c2ncc(-c3cnc(N)c(OCc4cccc(C#CC(C)(C)O)c4)c3)s2)CC1]

### TECHNICAL FIELD

The present invention relates to the technical field of chemical pharmaceutics, and in particular to use of a compound (4-(3-(((2-amino-5-(2-(1-methylpiperidin-4-yl)thiazol-5-yl)pyridin-3-yl)oxy)methyl) phenyl)-2-methylbut-3-yn-2-ol) in preventing and/or treating pathogen (particularly, virus, such as coronavirus) infection in animals.

### BACKGROUND

Viral diseases are a group of diseases doing most serious harm to animals in veterinary practice, and particularly, the prevalence of some highly infectious viral diseases often cause enormous economic and mental losses to the breeders. Viral diseases in animals usually spread quickly, feature high morbidity rate and mortality rate, and seriously endanger the health and life of animals. Moreover, some viruses can be transmitted to human beings through animals, threatening the safety of human beings.

Viral diseases in animals can be caused by a wide variety of viruses, for example, viruses being of Herpesviridae, Rhabdoviridae, Reoviridae, Poxviridae, Asfarviridae, Adenoviridae, Parvoviridae, Circoviridae, Orthomyxoviridae, Paramyxoviridae and Coronaviridae. Coronaviruses are a large family of viruses widely present in the nature and are named for their morphological similarity to crown under an electron microscope. They mainly cause respiratory diseases and can infect various mammals, such as pigs, cattle, cats, dogs, minks and camels, and various birds. For example, porcine deltacoronavirus (PDCoV) is a novel porcine enteric coronavirus that can cause diarrhea and vomiting, rapid dehydration and death of organ failure in piglets of 5-15 days old, with morbidity and mortality rates up to 50% to 100%; avian infectious bronchitis is an acute highly contagious respiratory infectious diseases in chicken caused by infectious bronchitis virus and is clinically characterized by dyspnea, rales, cough, mouth breathing and sneezing, a non-nephropathogenic strain that does not cause complication generally leads to a low mortality, but to reduced egg production and quality in laying hens; bovine coronavirus (BCV) is a pathogenic virus of cattle, is generally regarded as an important pathogen of neonatal calf diarrhea at present, and can cause respiratory tract infection in cattle and winter bloody dysentery in adult cattle; the epidemic diarrhea (coronavirus enteritis) in minks, foxes and raccoon dogs is caused by coronavirus except for mink parvovirus enteritis, and infected dogs, foxes, raccoon dogs and minks usually demonstrate hemorrhagic gastroenteritis symptoms and are susceptible to cluster epidemic diarrhea, showing a high spreading speed, a mortality more than 30% and a higher morbidity in animals giving birth in the year is higher than in stud animals.

Although many vaccines against viral diseases have been developed with the development of pharmaceutical technology, virus mutants impose a burden of efficacy evaluation of the vaccines against the emerging viruses as well as a financial burden, and the morbidity and mortality of viral diseases still remain high, leaving a great demand for drugs with good antiviral efficacy to the field of veterinary.

### SUMMARY

The present invention provides use of compound 4-(3-(((2-amino-5-(2-(1-methylpiperidin-4-yl)thiazol-5-yl)pyridin-3-yl)oxy)methyl) phenyl)-2-methylbut-3-yn-2-ol (having a structure of formula I below) or a pharmaceutically acceptable salt, a stereoisomer, an ester, a prodrug, a solvate or a deuterated compound thereof in preparing a medicament for preventing and/or treating a disease or condition caused by or associated with pathogen infection in an animal

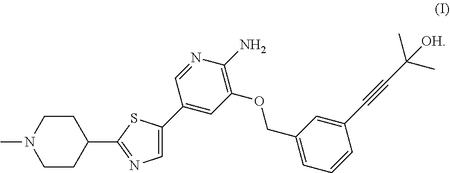 [SMILES: CN1CCC(c2ncc(-c3cnc(N)c(OCc4cccc(C#CC(C)(C)O)c4)c3)s2)CC1]

Specifically, the pathogen described herein may be a microorganism, a parasite (a protozoon, a worm, etc.) or any other vector; specifically, the above microorganism described above can be selected from one or more of a virus, a chlamydia, a rickettsia, a mycoplasma, a bacterium, a spirochete, a fungus and the like.

In one embodiment of the present invention, the pathogen described above is a virus. Specifically, the virus described above may be a species of Herpesviridae (e.g., pseudorabies virus, bovine infectious rhinotracheitis virus, Marek's disease virus, avian infectious laryngotracheitis virus, duck plague virus), Iridoviridae (e.g., lymphocystis disease virus), Baculoviridae (e.g., baculovirus penaei), Rhabdoviridae (e.g., rabies virus, rhabdovirus carpio), Reoviridae (e.g., avian orthoreovirus, bluetongue virus, cytoplasmic polyhedrosis virus), Birnaviridae (e.g., infectious bursal disease virus), Poxviridae (e.g., sheep pox virus, goat pox virus, myxomatosis virus), Asfarviridae (e.g., African swine fever virus), Adenoviridae (e.g., canine infectious hepatitis virus, egg drop syndrome virus), Parvoviridae (e.g., porcine parvovirus, canine parvovirus, goose parvovirus, feline panleukopenia virus, mink enteritis virus, mink Aleutian disease virus), Circoviridae (e.g., porcine circovirus), Retroviridae (e.g., avian leukosis virus, caprine arthritis/encephalomyelitis virus, equine infectious anemia virus), Orthomyxoviridae (e.g., avian influenza virus), Paramyxoviridae (e.g., Newcastle disease virus, canine distemper virus, cattle plague virus), Coronaviridae, Arteriviridae (e.g., porcine reproductive and respiratory syndrome virus), Picornaviridae (e.g., foot-and-mouth disease virus, swine vesicular disease virus, duck hepatitis virus), Caliciviridae (e.g., rabbit hemorrhagic disease virus), Flaviviridae (e.g., swine fever virus, bovine viral diarrhea virus, Japanese encephalitis virus), a prion, Filoviridae (e.g., Marburg virus, Ebola virus), Polyomaviridae, Papillomaviridae, Nimaviridae, Retroviridae, Hepadnaviridae, Papovaviridae, Bornaviridae, Bunyaviridae, Arenaviridae, Roniviridae, Hepeviridae, Astroviridae, Togaviridae, Dicistroviridae, Nodaviridae and the like.

Specifically, the virus described above may be, for example, avian infectious bronchitis virus (IBV), porcine transmissible gastroenteritis virus (TGEV), porcine epidemic diarrhea virus (PEDV), porcine hemagglutinating encephalomyelitis virus (HEV), mouse hepatitis virus (MHV), turkey bluecomb virus (TCDV), bovine coronavirus (BCV), canine coronavirus (CCV), feline infectious peritonitis virus (FIPV), rat coronavirus (RCV), rat sialodacryoadenitis coronavirus (SDAV), mink epidemic diarrhea coronavirus, sheep pox virus, goat pox virus, bovine lumpy skin disease virus, fowl pox virus, feline pox virus, infectious pustule virus, rabbit myxomatosis virus, African swine fever virus, pseudorabies virus, porcine cytomegalovirus, avian infectious laryngotracheitis virus, duck plague virus, feline viral rhinotracheitis virus, canine herpes virus, bovine infectious rhinotracheitis virus, equine rhinopneumonitis virus, Marek's disease virus, malignant catarrhal fever virus, porcine adenovirus, canine viral hepatitis virus, feline adenovirus, porcine parvovirus, feline panleukopenia (feline distemper, feline infectious enteritis) virus, gosling plague virus, canine parvovirus, Muscovy duck parvovirus, porcine circovirus, chicken infectious anemia virus, avian leukosis virus, feline leukemia virus, bovine leukemia virus, feline immunodeficiency virus, Maedi-visna disease virus, caprine viral arthritis-encephalitis virus, equine infectious anemia virus, bovine immunodeficiency virus, feline syncytium-forming virus (foamy virus), avian viral arthritis virus, bluetongue virus, Ibaraki disease virus, Chuzan virus, African horse sickness virus, rotavirus, infectious bursal disease virus, canine viral papilloma virus, feline viral papilloma virus, Nipah disease virus, Hendra disease virus, porcine blue eye disease virus, canine parainfluenza virus, avian paramyxovirus, chicken Newcastle disease virus, canine distemper virus, peste des petits ruminants virus, cattle plague virus, avian pneumovirus, avian mumps virus (measles virus), feline paramyxovirus, rabies virus, vesicular stomatitis virus, bovine ephemeral fever/three day fever/transient fever virus, Borna virus, influenza virus, Rift Valley fever virus, Akabane virus, Hantavirus, feline enteric coronavirus, porcine hemagglutinating encephalomyelitis virus, porcine reproductive and respiratory syndrome virus, equine viral arteritis virus, foot-and-mouth disease virus, swine vesicular disease virus, porcine enterovirus, duck viral hepatitis virus, avian encephalomyelitis virus, encephalomyocarditis virus, vesicular exanthema of swine virus, feline calicivirus, rabbit viral hemorrhagic disease virus, canine hepatitis E virus, Getah virus, Japanese encephalitis B/epidemic encephalitis B virus, forest encephalitis virus, duck flavivirus, swine fever virus, bovine viral diarrhea-mucosal disease virus, border disease virus and the like.

In one embodiment of the present invention, the virus described above is a coronavirus, specifically, avian infectious bronchitis virus (IBV), porcine transmissible gastroenteritis virus (TGEV), porcine epidemic diarrhea virus (PEDV), porcine hemagglutinating encephalomyelitis virus (HEV), mouse hepatitis virus (MHV), turkey bluecomb virus (TCDV), bovine coronavirus (BCV), canine coronavirus (CCV), feline infectious peritonitis virus (FIPV), rat coronavirus (RCV), rat sialodacryoadenitis coronavirus (SDAV), mink epidemic diarrhea coronavirus and the like.

In one embodiment of the present invention, the virus described above is a feline infectious peritonitis virus.

As used herein, the term “animal” refers to a non-human animal, particularly to a vertebrate, and specifically to, e.g., a mammal (for example, pig, cattle, sheep, horse, donkey, dog, cat, rabbit, rodent, fox, raccoon dog, mink, camel), fish, bird (for example, chicken, duck, goose, pigeon, quail, parrot, etc.), amphibian and reptile, unless otherwise indicated. Particularly, the animal described above is a domestic animal, i.e., an animal raised and domesticated by humans with artificially controlled reproduction for purposes such as food, labor, fur, companionship and experiment, such as an economic animal, a companion animal and a laboratory animal.

In one embodiment of the present invention, in the use described above, the animal is an economic animal, such as livestock (e.g., pig, cattle, sheep, horse, donkey, fox, raccoon dog, mink and camel), poultry (chicken, duck, goose, pigeon, quail, etc.).

In one embodiment of the present invention, in the use described above, the animal is a companion animal, such as dog, cat, rabbit, rodent (e.g., guinea pig, hamster, gerbil, chinchilla and squirrel), fish, pigeon and parrot.

In one embodiment of the present invention, in the use described above, the animal is a laboratory animal, such as monkey, dog, rabbit, cat, rodent, etc.

Specifically, the disease or condition described above may include avian infectious bronchitis, porcine transmissible gastroenteritis, porcine epidemic diarrhea, canine coronavirus disease, porcine hemagglutinating encephalomyelitis, hepatitis, encephalitis and enteritis caused by mouse hepatitis virus, turkey bluecomb, neonatal calf diarrhea, bovine blood dysentery, feline infectious peritonitis, rat sialodacryoadenitis, mink epidemic diarrhea, sheep pox, goat pox, bovine lumpy skin disease, fowl pox, feline pox, infectious pustule, rabbit myxomatosis, African swine fever, pseudorabies, porcine cytomegalovirus infection, avian infectious laryngotracheitis, duck plague, feline viral rhinotracheitis, canine herpes virus infection, bovine infectious rhinotracheitis, equine rhinopneumonitis, Marek's disease, malignant catarrhal fever, porcine adenovirus infection, canine viral hepatitis, feline adenovirus disease, porcine parvovirus disease, feline panleukopenia (feline distemper, feline infectious enteritis), gosling plague, canine parvovirus disease, Muscovy duck parvovirus disease, porcine circovirus disease, chicken infectious anemia, avian leukemia, feline leukemia, bovine leukemia, feline immunodeficiency disease, Maedi-visna disease, caprine viral arthritis-encephalitis, equine infectious anemia, bovine immunodeficiency virus infection, feline syncytium-forming virus (foamy virus) infection, avian viral arthritis, bluetongue, Ibaraki disease, Chuzan disease, African horse sickness, rotavirus disease, infectious bursal disease, canine viral papilloma, feline viral papilloma, Nipah disease, Hendra disease, porcine blue eye disease, canine parainfluenza virus infection, avian paramyxovirus infection, chicken Newcastle disease, canine distemper, peste des petits ruminants, cattle plague, avian pneumovirus infection, avian mumps virus infection (measles virus infection), feline paramyxovirus disease, rabies, vesicular stomatitis, bovine ephemeral fever/three day fever/transient fever, Borna disease, influenza, Rift Valley fever, Akabane disease, hantavirus disease, feline enteric coronavirus infection, porcine hemagglutinating encephalomyelitis, porcine reproductive and respiratory syndrome, equine viral arteritis, foot-and-mouth disease, swine vesicular disease, porcine enterovirus infection, duck viral hepatitis, avian encephalomyelitis, encephalomyocarditis, vesicular exanthema of swine, feline calicivirus disease, rabbit viral hemorrhagic disease, canine hepatitis E, Getah virus disease, Japanese encephalitis B/epidemic encephalitis B, forest encephalitis, duck flavivirus infection, swine fever, bovine viral diarrhea-mucosal disease, border disease and the like.

In one embodiment of the present invention, the disease or condition described above is selected from avian infectious bronchitis, porcine transmissible gastroenteritis, porcine epidemic diarrhea, canine coronavirus disease, porcine hemagglutinating encephalomyelitis, hepatitis, encephalitis and enteritis caused by mouse hepatitis virus, turkey bluecomb, neonatal calf diarrhea, bovine blood dysentery, feline infectious peritonitis, rat sialodacryoadenitis and mink epidemic diarrhea.

In one embodiment of the present invention, the disease described above is feline infectious peritonitis.

The present invention further provides use of a compound of formula I or a pharmaceutically acceptable salt, a stereoisomer, an ester, a prodrug, a solvate or a deuterated compound thereof in preparing a product for resisting pathogen infection in an animal.

Specifically, in the use described above, the pathogen and the animal have the corresponding definitions described above in the present invention.

Specifically, the product described above may be used for either therapeutic purposes or non-therapeutic purposes.

In one embodiment of the present invention, the product described above is a pharmaceutical composition.

Specifically, in the pharmaceutical composition described above, the compound of formula I described above may be used as the sole active ingredient or may be used in combination with one or more additional active ingredients for the same indication or different indications, wherein the heterocyclic compound described above and the additional active ingredients may be formulated for simultaneous, separate or sequential administration.

Specifically, the pharmaceutical composition described above further comprises a pharmaceutically acceptable excipient, in particular an excipient acceptable in the field of veterinary.

Specifically, the pharmaceutical composition described above may be in any dosage form or administration form, which can be selected by those skilled in the art according to concrete circumstances. For example, the administration form can be, but is not limited to, oral, sublingual, inhalational, subcutaneous, intramuscular, intravenous, intraperitoneal, intra-organ, intranasal, intrarectal, transdermal, ocular or rectal form; the dosage form can be, but is not limited to, tablets, pills, powders, granules, capsules, lozenges, syrups, solutions, emulsions, suspensions, controlled release formulations, aerosols, films, injections, intravenous drip infusions, transdermal absorption formulations, ointments, lotions, adhesive formulations, suppositories, nasal formulations, pulmonary formulations, eye drops and the like. In one embodiment of the present invention, the pharmaceutical composition described above is an injection.

The various forms of the pharmaceutical composition described above can be prepared according to conventional production methods in the field of veterinary drugs.

In one embodiment of the present invention, the product described above is a functional food composition.

Specifically, in the functional food composition described above, the compound of formula I may be used as the sole active ingredient, or may be used in combination with one or more additional active ingredients.

Specifically, the functional food composition described above may further comprise an animal food excipient.

Specifically, the functional food composition described above may be in any form, such as tablets, pills, capsules, candies (e.g., tablet candies, gel candies, gum-based candies, etc.), solid beverages (e.g., powders, granules, etc.), liquid beverages and the like.

The various forms of the functional food composition described above can be prepared according to conventional production methods in the field of veterinary functional food.

In one embodiment of the present invention, the product described above is an additive, which can be added in a small amount or in a trace amount during the production, processing and use of the animal food (e.g., livestock and poultry feed, pet daily feed, pet snack and the like).

The present invention further provides a method for preventing and/or treating a disease or condition caused by or associated with pathogen infection in an animal, comprising: administering to a subject an effective amount of the compound of formula I or the pharmaceutically acceptable salt, the stereoisomer, the ester, the prodrug, the solvate or the deuterated compound thereof described above in the present invention.

Specifically, in the method described above, the pathogen, the animal, and the disease or condition has the corresponding definition described above in the present invention.

The inventor of the present invention found through experiments that the compound can effectively treat diseases (such as feline infectious peritonitis) caused by pathogen (especially virus, such as coronavirus) infection in animals, improves the survival conditions of the animals, and has better commercial value and application prospect in the field of veterinary anti-pathogen (especially virus, such as coronavirus) therapies.

### BRIEF DESCRIPTION OF THE DRAWINGS

FIG. 1 illustrates chest X-ray images of an affected cat in Example 1 of the present invention.

FIG. 2 illustrates a curve of clinical survival in Example 2 of the present invention, wherein the administration day was taken as the first day.

### DETAILED DESCRIPTION

Unless otherwise defined, all scientific and technical terms used herein have the same meaning as commonly understood by those skilled in the art to which the present invention relates.

The term “pathogen” refers to a microorganism (including virus, chlamydia, rickettsia, mycoplasma, bacterium, spirochete, fungus and the like), a parasite (protozoon, worm and the like) or any other vector that can cause infection or diseases in humans or animals and plants. Among these, the bacterium may be, for example, a Gram-positive cocci, an enterobacterium, a vibrio, a pasteurella, a Gram-negative aerobic bacterium, a Gram-negative microaerophilic and anaerobic bacterium, a Gram-positive non-spore-forming bacillus, a Gram-positive spore-forming bacillus and mycobacterium; the fungus can be, for example, a candida, a cryptococcus and an aspergillus. In the present invention, the pathogen generally refers to a microorganism, a parasite or any other vector that can cause infection or diseases in animals. In one embodiment of the present invention, the pathogen is a virus.

The term “viral infection” refers to the process by which a virus invades the body through a variety of pathways and proliferates in susceptible host cells. The infected body may demonstrate different clinical types. The infection can be classified into apparent infection and latent infection according to the presence or absence of symptoms. An infection where the virus proliferates in host cells but no remarkable clinical symptoms are observed due to the small amount or weak toxicity of the invading virus or the strong resistance of the body is referred to as latent infection.

Although a latent infection does not demonstrate clinical symptoms, the virus proliferates in vivo and the body spreads new viruses to others and becomes an important source of infection. Therefore, resisting viral infection is also required for a host with latent infection. An infection where the virus proliferates in host cells and causes remarkable clinical symptoms due to the large amount or strong toxicity of the invading virus or the weak resistance of the body is referred to as latent infection.

As used herein, the term “animal” refers to a non-human animal, particularly to a vertebrate, and specifically to, e.g., a mammal (for example, pig, cattle, sheep, horse, donkey, dog, cat, rabbit, rodent, fox, raccoon dog, mink, camel), fish, bird (for example, chicken, duck, goose, pigeon, quail, parrot, etc.), amphibian and reptile, unless otherwise indicated. Particularly, the animal described above is a domestic animal, i.e., an animal raised and domesticated by humans with artificially controlled reproduction for purposes such as food, labor, fur, companionship and experiment, such as an economic animal, a companion animal and a laboratory animal. The term “economic animal” refers to an animal, such as livestock, poultry and the like, that is raised for meat, milk, fur, labor or other economic purposes; the term “livestock” refers to a domestic animal that is raised and utilized by humans for breeding, and is beneficial to agricultural production; the term “poultry” refers to an avian animal that is fed by humans mainly for meat, eggs, feather or other purposes; the term “companion animal” refers to an animal raised for mental purposes (e.g., for appreciation and companion purposes) rather than economic purposes; the term “laboratory animal” refers to an animal raised for scientific application purposes. The term “salt” is to be understood as any form of the compound according to the present invention, wherein the compound is in an ionic form, is charged and coupled with an oppositely charged ion (cation or anion), or is in a solution. Also included within this definition are quaternary ammonium salts and complexes of the molecule with other molecules and ions, particularly complexes formed by ionic interactions. The term “solvate” is to be understood as any form of the compound of the present invention, wherein the compound is linked to another molecule (usually a polar solvent) by a non-covalent bond, particularly including hydrate and alcoholate such as methanolate. Particularly, the solvate is hydrate.

The term “prodrug” is used in its broad sense and encompasses derivatives that can be converted into the compound of the present invention in vivo. Examples of prodrugs include, but are not limited to, derivatives and metabolites of the compound, including biohydrolyzable moieties such as biohydrolyzable amides, biohydrolyzable esters, biohydrolyzable carbamates, biohydrolyzable carbonates, biohydrolyzable ureides, and biohydrolyzable phosphate analogs. Preferably, the prodrug having a carboxyl functional group is a lower alkyl ester of a carboxylic acid. The carboxylic acid ester is readily esterified from any carboxylic acid moiety present in the molecule. Prodrugs can generally be prepared by known methods, such as those described in “Burger's Medicinal Chemistry and Drug Discovery”, sixth edition (Donald J. Abraham ed., 2001, Wiley) and “Design and Applications of Prodrugs” (H. Bundgaard ed., 1985, Harwood Academic Publishers).

Any compound referred to herein is intended to represent such particular compound and certain variations or certain forms thereof. Particularly, the compounds referred to herein may have asymmetric centers and thus have different enantiomeric or diastereomeric forms. Thus, any given compound referred to herein represents any one of racemates thereof, one or more enantiomeric forms, one or more diastereomeric forms and mixtures thereof. Likewise, molecules with a double bond may also have stereoisomers or geometric isomers. Thus, in some cases, an (E)-isomer or (Z)-isomer (trans and cis isomers) may be present. If a molecule contains multiple double bonds, each double bond will have its own stereoisomerism, which may be the same with or be different from the stereoisomerisms of other double bonds of the molecule. In addition, the compounds referred to herein may have atropisomers. All stereoisomers of the compounds referred to herein, including enantiomers, diastereomers, geometric isomers, atropisomers and mixtures thereof, are within the scope of the present invention. Unless otherwise specified, the compounds referred to herein may also include isotope-labeled forms, i.e., compounds that differ only in the presence of one or more isotope atoms. For example, compounds of existing structures with at least one hydrogen atom replaced by only deuterium or tritium, at least one carbon replaced by a ¹³C or ¹⁴C-enriched carbon, or at least one nitrogen replaced by a ¹⁵N-enriched nitrogen are included within the scope of the present invention.

The compounds described herein or the salts, the solvates, the stereoisomers, the ethers or the esters thereof are preferably in a pharmaceutically acceptable form. The term “pharmaceutically acceptable” refers to that the molecular and compositions containing the same do not produce adverse, allergic or other untoward reactions when properly administered to a subject.

The technical schemes of the present invention will be clearly and completely described below with reference to the examples of the present invention, and it is obvious that the described examples are only a part of the examples of the present invention but not all of them. Based on the examples of the present invention, all other examples obtained by those of ordinary skill in the art without creative work shall fall within the protection scope of the present invention.

### Example 1

1. Methodology

A female British shorthair cat (Lele) of 2.5 kg, aged 8 month, unneutered. The cat had symptoms of increased body temperature, mental depression, decreased appetite, and increased abdominal girth in January, 2020. After 1 week, the cat was admitted. On January 18, the cat was diagnosed with feline infectious peritonitis (FIP) positive. After being treated with GS-441524 for 3 days, the cat was then administered with furosemide, Synulox®, prednisolone and itraconazole (P.O., q24h, 10 mg/kg) for 1 month. The drugs were discontinued after ascites regressed. Early in April, the disease recurred, showing decreased food intake and listlessness. X-ray examination suggested hydrothorax, and PCR test showed feline coronavirus positive (the CT value was 25.11). The cat was enrolled on April 8, and was treated with the drug of the present invention at 2.0 mg/kg (SC, q24h) on days 1-14 (D1-14). Proper supportive treatments were given at the same time, including adjuvant treatment such as Moringa (a hepatoprotective drug), Lysinphirst (lysine) and Doctor Dolittle® (lactoferrin).

2. Preparation and Use

The pharmaceutically active compound described above is a pure powder have the following structure. The compound was added to a 0.9% normal saline (50 mL of endotoxin-free water and 0.45 g of NaCl), and the mixture was uniformly mixed and filtered through a filter membrane to give a clarified solution. The diluted compound was stored in a plastic-sealed sterile vial, stored in a refrigerator at 4° C., slowly heated to room temperature before injection, and administered using a disposable syringe. The injection ranged across the back, starting from 2 cm posterior to the scapula to the middle of the lumbar spine, half of the distance of the adjacent chest to flank.

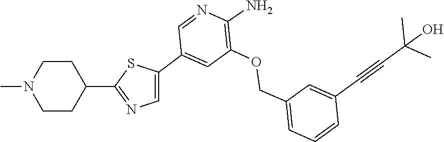 [SMILES: CN1CCC(c2ncc(-c3cnc(N)c(OCc4cccc(C#CC(C)(C)O)c4)c3)s2)CC1]

3. Results

The treatment was given by injection in the morning every day. Body temperature, appetite, mental state and respiratory state were measured and assessed at 15:00 pm, and urination and defecation conditions were recorded. The results are shown in Table 1.

TABLE 1
Experimental results

| | | Body | Body | | | | | | |
|---|---|---|---|---|---|---|---|---|---|
| | | temperature | temperature | Mental | Food | Respiratory | Defecation | | |
| No. | Time | Morning | Afternoon | state | intake | state | condition | Notes | |
| D 0 | 4-8 | | 38.5 | Listless - 3 | Decreased | Normal - 5 | Normal - 5 | 2.5 | kg body weight |
| | | | | | intake - 3 | | | | at enrollment |
| D 1 | 4-9 | | 38.6 | Slightly | Decreased | Normal | Normal | | |
| | | | | improved - 3.5 | intake - 3 | | | | |
| D 2 | 4-10 | | 38.1 | Improved - 4 | Improved - 4 | Normal | Normal | | |
| D 3 | 4-11 | | 38 | Good - 5 | Good - 5 | Normal | Normal | | |
| D 4 | 4-12 | | 38.3 | Good | Good | Normal | Normal | | |
| D 5 | 4-13 | | 38.3 | Good | Good | Normal | Normal | | |
| D 6 | 4-14 | | 38.3 | Good | Good | Normal | Normal | | |
| D 7 | 4-15 | | 38.1 | Good | Good | Normal | Normal | 2.75 | kg body weight |
| D 8 | 4-16 | | 38 | Good | Good | Normal | Normal | | |
| D 9 | 4-17 | | 38.2 | Good | Good | Normal | Normal | | |
| D 10 | 4-18 | | 38.3 | Good | Good | Normal | Normal | | |
| D 11 | 4-19 | | 38.4 | Good | Good | Normal | Normal | | |
| D 12 | 4-20 | | 38.5 | Good | Good | Normal | Normal | | |
| D 13 | 4-21 | 38.9 | 38.5 | Good | Good | Normal | Normal | | |
| D 14 | 4-22 | 38.4 | 38.4 | Good | Good | Normal | Normal | 2.75 | kg body weight |

The following procedures were performed on DO, D7 and D14 after enrollment: 1 mL of serum sample was collected and frozen for multi-factor assay. The results are shown in Table 2.

TABLE 2
Complete blood count results

| Complete blood count | | Normal range | D0 | D7 | D14 |
|---|---|---|---|---|---|
| WBC | White blood cell | 5.5~19.5 | 13.5 | 18.7 | 19.7 |
| RBC | Red blood cell | 5.0~10.0 | 10.5 | 7.94 | 7.94 |
| HGB | Hemoglobin | 80~150 | 117 | 94 | 91 |
| HCT | Hematocrit | 24.0~45.0 | 37.5 | 28.9 | 29.9 |
| MCV | Mean corpuscular | 39.0~55.0 | 35.9 | 36.4 | 37.7 |
| | volume | | | | |
| MCH | Mean corpuscular | 12.5~17.5 | 11.2 | 11.8 | 11.5 |
| | hemoglobin | | | | |

3) Chest X-Ray

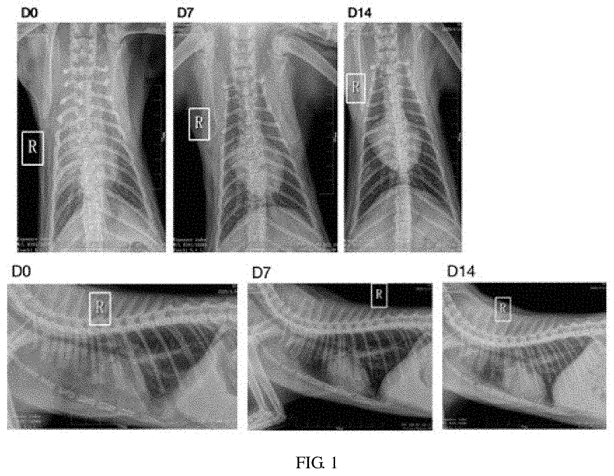

As shown in FIG. 1.

4) Analysis of Results

The above results suggested wet FIP (in the chest), showing hydrothorax, listlessness and anorexia, which were typical clinical symptoms of feline infectious peritonitis. Because the animal had no fever symptom at enrollment, the fever alleviation effect cannot be observed during treatment, and the body temperature was always maintained at in the normal range of 38-38.5° C. throughout the treatment. After 2 weeks of treatment the cat demonstrated remarkable clinical response with improved appetite and mental state, and weight gain. The hydrothorax completely regressed since about 7 days after the treatment. The body weight increased by 9% during and after the treatment.

### Example 2

A total of 15 cats with clinically confirmed FIP were enrolled, with hydrothorax/ascites, positive CT value by PCR, and no neurological symptoms. 8 cats were allocated into the control group, and 7 cats were allocated into the treatment group.

The groups were pre-treated with GS-441524 for 3 days. For the control group, necessary supportive treatment was given on D1—D14. Group A animals were observed for 3 days, necessary supportive treatment and the drug of the present invention (2.0 mg/kg, SC, q24h) were given on D1—D14.

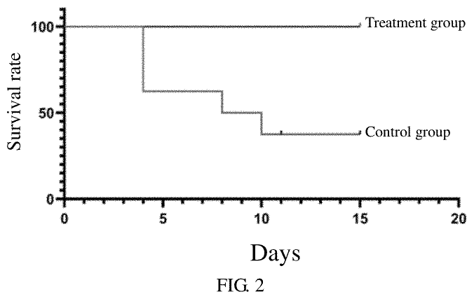

The occurrence of death or neurological symptoms was judged as clinical death, and the clinical survival curve was plotted after 14 days. The results are shown in FIG. 2.

The Log Rank (Mantel-Cox) and Breslow (Generalized Wilcoxon) methods were used for validity test, and both methods indicated a significant difference (P<0.05). The above description is only for the purpose of illustrating the preferred example of the present invention, and is not intended to limit the scope of the present invention. Any modifications, equivalents and the like made without departing from the spirit and principle of the present invention should be included in the protection scope of the present invention.

The foregoing examples and methods described herein may vary based on the abilities, experience, and preferences of those skilled in the art.

The certain order in which the steps of the method are listed in the present invention does not constitute any limitation on the order of the steps of the method.

1. Use of a compound of formula I or a pharmaceutically acceptable salt, a stereoisomer, an ester, a prodrug, a solvate or a deuterated compound thereof in preparing a medicament for preventing and/or treating a disease or condition caused by or associated with pathogen infection in an animal

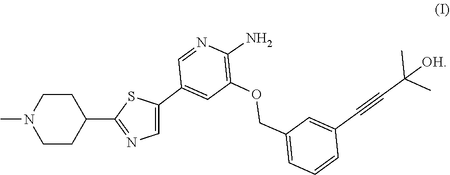 [SMILES: CN1CCC(c2ncc(-c3cnc(N)c(OCc4cccc(C#CC(C)(C)O)c4)c3)s2)CC1]

2. The use according to claim 1, wherein the pathogen is a virus;
preferably, the virus is a species selected from Herpesviridae, Iridoviridae, Baculoviridae, Rhabdoviridae, Reoviridae, Birnaviridae, Poxviridae, Asfarviridae, Adenoviridae, Parvoviridae, Circoviridae, Retroviridae, Orthomyxoviridae, Paramyxoviridae, Coronaviridae, Arteriviridae, Picornaviridae, Caliciviridae, Flaviviridae, a prion, Filoviridae, Polyomaviridae, Papillomaviridae, Nimaviridae, Retroviridae, Hepadnaviridae, Papovaviridae, Bornaviridae, Bunyaviridae, Arenaviridae, Roniviridae, Hepeviridae, Astroviridae, Togaviridae, Dicistroviridae and Nodaviridae;
preferably, the virus is a coronavirus.

3. The use according to claim 1, wherein the virus is selected from avian infectious bronchitis virus, porcine transmissible gastroenteritis virus, porcine epidemic diarrhea virus, porcine hemagglutinating encephalomyelitis virus, mouse hepatitis virus, turkey bluecomb virus, bovine coronavirus, canine coronavirus, feline infectious peritonitis virus, rat coronavirus, rat sialodacryoadenitis coronavirus, mink epidemic diarrhea coronavirus, sheep pox virus, goat pox virus, bovine lumpy skin disease virus, fowl pox virus, feline pox virus, infectious pustule virus, rabbit myxomatosis virus, African swine fever virus, pseudorabies virus, porcine cytomegalovirus, avian infectious laryngotracheitis virus, duck plague virus, feline viral rhinotracheitis virus, canine herpes virus, bovine infectious rhinotracheitis virus, equine rhinopneumonitis virus, Marek's disease virus, malignant catarrhal fever virus, porcine adenovirus, canine viral hepatitis virus, feline adenovirus, porcine parvovirus, feline panleukopenia virus, gosling plague virus, canine parvovirus, Muscovy duck parvovirus, porcine circovirus, chicken infectious anemia virus, avian leukosis virus, feline leukemia virus, bovine leukemia virus, feline immunodeficiency virus, Maedi-visna disease virus, caprine viral arthritis-encephalitis virus, equine infectious anemia virus, bovine immunodeficiency virus, feline syncytium-forming virus, avian viral arthritis virus, bluetongue virus, Ibaraki disease virus, Chuzan virus, African horse sickness virus, rotavirus, infectious bursal disease virus, canine viral papilloma virus, feline viral papilloma virus, Nipah disease virus, Hendra disease virus, porcine blue eye disease virus, canine parainfluenza virus, avian paramyxovirus, chicken Newcastle disease virus, canine distemper virus, peste des petits ruminants virus, cattle plague virus, avian pneumovirus, avian mumps virus, feline paramyxovirus, rabies virus, vesicular stomatitis virus, bovine ephemeral fever/three day fever/transient fever virus, Borna virus, influenza virus, Rift Valley fever virus, Akabane virus, Hantavirus, feline enteric coronavirus, porcine hemagglutinating encephalomyelitis virus, porcine reproductive and respiratory syndrome virus, equine viral arteritis virus, foot-and-mouth disease virus, swine vesicular disease virus, porcine enterovirus, duck viral hepatitis virus, avian encephalomyelitis virus, encephalomyocarditis virus, vesicular exanthema of swine virus, feline calicivirus, rabbit viral hemorrhagic disease virus, canine hepatitis E virus, Getah virus, Japanese encephalitis B/epidemic encephalitis B virus, forest encephalitis virus, duck flavivirus, swine fever virus, bovine viral diarrhea-mucosal disease virus and border disease virus;
preferably, the virus is a coronavirus selected from avian infectious bronchitis virus, porcine transmissible gastroenteritis virus, porcine epidemic diarrhea virus, porcine hemagglutinating encephalomyelitis virus, mouse hepatitis virus, turkey bluecomb virus, bovine coronavirus, canine coronavirus, feline infectious peritonitis virus, rat coronavirus, rat sialodacryoadenitis coronavirus and mink epidemic diarrhea coronavirus;
more preferably, the virus is a feline infectious peritonitis virus.

4. The use according to claim 1, wherein the animal is a domestic animal;
preferably, the animal is an economic animal, a companion animal or a laboratory animal.

5. The use according to claim 1, wherein the disease or condition includes avian infectious bronchitis, porcine transmissible gastroenteritis, porcine epidemic diarrhea, canine coronavirus disease, porcine hemagglutinating encephalomyelitis, hepatitis, encephalitis and enteritis caused by mouse hepatitis virus, turkey bluecomb, neonatal calf diarrhea, bovine blood dysentery, feline infectious peritonitis, rat sialodacryoadenitis, mink epidemic diarrhea, sheep pox, goat pox, bovine lumpy skin disease, fowl pox, feline pox, infectious pustule, rabbit myxomatosis, African swine fever, pseudorabies, porcine cytomegalovirus infection, avian infectious laryngotracheitis, duck plague, feline viral rhinotracheitis, canine herpes virus infection, bovine infectious rhinotracheitis, equine rhinopneumonitis, Marek's disease, malignant catarrhal fever, porcine adenovirus infection, canine viral hepatitis, feline adenovirus disease, porcine parvovirus disease, feline panleukopenia, gosling plague, canine parvovirus disease, Muscovy duck parvovirus disease, porcine circovirus disease, chicken infectious anemia, avian leukemia, feline leukemia, bovine leukemia, feline immunodeficiency disease, Maedi-visna disease, caprine viral arthritis-encephalitis, equine infectious anemia, bovine immunodeficiency virus infection, feline syncytium-forming virus infection, avian viral arthritis, bluetongue, Ibaraki disease, Chuzan disease, African horse sickness, rotavirus disease, infectious bursal disease, canine viral papilloma, feline viral papilloma, Nipah disease, Hendra disease, porcine blue eye disease, canine parainfluenza virus infection, avian paramyxovirus infection, chicken Newcastle disease, canine distemper, peste des petits ruminants, cattle plague, avian pneumovirus infection, avian mumps virus infection, feline paramyxovirus disease, rabies, vesicular stomatitis, bovine ephemeral fever/three day fever/transient fever, Borna disease, influenza, Rift Valley fever, Akabane disease, hantavirus disease, feline enteric coronavirus infection, porcine hemagglutinating encephalomyelitis, porcine reproductive and respiratory syndrome, equine viral arteritis, foot-and-mouth disease, swine vesicular disease, porcine enterovirus infection, duck viral hepatitis, avian encephalomyelitis, encephalomyocarditis, vesicular exanthema of swine, feline calicivirus disease, rabbit viral hemorrhagic disease, canine hepatitis E, Getah virus disease, Japanese encephalitis B/epidemic encephalitis B, forest encephalitis, duck flavivirus infection, swine fever, bovine viral diarrhea-mucosal disease and border disease;
more preferably, the disease is feline infectious peritonitis.

6. Use of a compound of formula I or a pharmaceutically acceptable salt, a stereoisomer, an ester, a prodrug, a solvate or a deuterated compound thereof in preparing a product for resisting pathogen infection in an animal

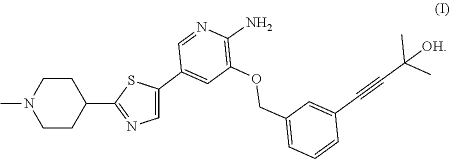 [SMILES: CN1CCC(c2ncc(-c3cnc(N)c(OCc4cccc(C#CC(C)(C)O)c4)c3)s2)CC1]

7. The use according to claim 6, wherein the pathogen is a virus;
preferably, the virus is a species selected from Herpesviridae, Iridoviridae, Baculoviridae, Rhabdoviridae, Reoviridae, Birnaviridae, Poxviridae, Asfarviridae, Adenoviridae, Parvoviridae, Circoviridae, Retroviridae, Orthomyxoviridae, Paramyxoviridae, Coronaviridae, Arteriviridae, Picornaviridae, Caliciviridae, Flaviviridae, a prion, Filoviridae, Polyomaviridae, Papillomaviridae, Nimaviridae, Retroviridae, Hepadnaviridae, Papovaviridae, Bornaviridae, Bunyaviridae, Arenaviridae, Roniviridae, Hepeviridae, Astroviridae, Togaviridae, Dicistroviridae and Nodaviridae;
preferably, the virus is a coronavirus.

8. The use according to claim 6, wherein the coronavirus is selected from avian infectious bronchitis virus, porcine transmissible gastroenteritis virus, porcine epidemic diarrhea virus, porcine hemagglutinating encephalomyelitis virus, mouse hepatitis virus, turkey bluecomb virus, bovine coronavirus, canine coronavirus, feline infectious peritonitis virus, rat coronavirus, rat sialodacryoadenitis coronavirus, mink epidemic diarrhea coronavirus, sheep pox virus, goat pox virus, bovine lumpy skin disease virus, fowl pox virus, feline pox virus, infectious pustule virus, rabbit myxomatosis virus, African swine fever virus, pseudorabies virus, porcine cytomegalovirus, avian infectious laryngotracheitis virus, duck plague virus, feline viral rhinotracheitis virus, canine herpes virus, bovine infectious rhinotracheitis virus, equine rhinopneumonitis virus, Marek's disease virus, malignant catarrhal fever virus, porcine adenovirus, canine viral hepatitis virus, feline adenovirus, porcine parvovirus, feline panleukopenia virus, gosling plague virus, canine parvovirus, Muscovy duck parvovirus, porcine circovirus, chicken infectious anemia virus, avian leukosis virus, feline leukemia virus, bovine leukemia virus, feline immunodeficiency virus, Maedi-visna disease virus, caprine viral arthritis-encephalitis virus, equine infectious anemia virus, bovine immunodeficiency virus, feline syncytium-forming virus, avian viral arthritis virus, bluetongue virus, Ibaraki disease virus, Chuzan virus, African horse sickness virus, rotavirus, infectious bursal disease virus, canine viral papilloma virus, feline viral papilloma virus, Nipah disease virus, Hendra disease virus, porcine blue eye disease virus, canine parainfluenza virus, avian paramyxovirus, chicken Newcastle disease virus, canine distemper virus, peste des petits ruminants virus, cattle plague virus, avian pneumovirus, avian mumps virus, feline paramyxovirus, rabies virus, vesicular stomatitis virus, bovine ephemeral fever/three day fever/transient fever virus, Borna virus, influenza virus, Rift Valley fever virus, Akabane virus, Hantavirus, feline enteric coronavirus, porcine hemagglutinating encephalomyelitis virus, porcine reproductive and respiratory syndrome virus, equine viral arteritis virus, foot-and-mouth disease virus, swine vesicular disease virus, porcine enterovirus, duck viral hepatitis virus, avian encephalomyelitis virus, encephalomyocarditis virus, vesicular exanthema of swine virus, feline calicivirus, rabbit viral hemorrhagic disease virus, canine hepatitis E virus, Getah virus, Japanese encephalitis B/epidemic encephalitis B virus, forest encephalitis virus, duck flavivirus, swine fever virus, bovine viral diarrhea-mucosal disease virus and border disease virus;
preferably, the virus is a coronavirus selected from avian infectious bronchitis virus, porcine transmissible gastroenteritis virus, porcine epidemic diarrhea virus, porcine hemagglutinating encephalomyelitis virus, mouse hepatitis virus, turkey bluecomb virus, bovine coronavirus, canine coronavirus, feline infectious peritonitis virus, rat coronavirus, rat sialodacryoadenitis coronavirus and mink epidemic diarrhea coronavirus;
more preferably, the virus is a feline infectious peritonitis virus.

9. The use according to claim 6, wherein the animal is a domestic animal;
preferably, the animal is an economic animal, a companion animal or a laboratory animal.

10. The use according to claim 6, wherein the product is a pharmaceutical composition, a functional food composition or an additive;
preferably, the pharmaceutical composition further comprises a pharmaceutically acceptable excipient;
preferably, the pharmaceutical composition is an injection.
